(19) United States
(12) Patent Application Publication
(10) Pub. No.: US 2023/0201116 A1
(43) Pub. Date: Jun. 29, 2023

(54) FORUMULATION OF MONODISPERSE KINETICALLY FROZEN POLYMER MICELLES VIA EQUILIBRATION-NANOPRECIPITATION

(71) Applicant: Purdue Research Foundation, West Lafayette, IN (US)

(72) Inventors: You-Yeon Won, West Lafayette, IN (US); Daniel James Fesenmeier, West Lafayette, IN (US)

(73) Assignee: Purdue Research Foundation, West Lafayette, IN (US)

(21) Appl. No.: 17/926,105

(22) PCT Filed: Jun. 1, 2021
(86) PCT No.: PCT/US2021/035087

§ 371 Date: Nov. 17, 2022

Related U.S. Application Data
(60) Provisional application No. 63/033,287, filed on Jun. 2, 2020.

(51) Int. Cl. A61K 9/107; A61K 47/34

(52) U.S. Cl. CPC A61K 9/1075; A61K 47/34

(57) ABSTRACT

A formulation and method of micelle production including the steps of dissolving amphiphilic block copolymers in a mixed solvent comprising water and a non-aqueous co-solvent, conducting a single-step dialysis against water or saline in order to produce monodisperse kinetically frozen polymer micelles with DLS size polydispersities less than about 0.2 in aqueous conditions or conducting an evaporation process for removal of non-aqueous solvent content in order to produce monodisperse kinetically frozen polymer micelles with DLS size polydispersities less than about 0.2 in aqueous conditions.

### FIELD

The present disclosure relates to production of monodisperse kinetically frozen polymer micelles in aqueous conditions.

### BACKGROUND

Acute Respiratory Distress Syndrome (ARDS) is a debilitating condition affecting 190,000 patients in the United States each year. ARDS occurs when the function of native lung surfactant becomes impaired leading to severe decrease in blood oxygenation. There are currently no therapeutic surfactant formulations which have been shown to treat this condition. The polymer formulation has been shown to be a promising candidate for lung surfactant replacement therapy as it forms a stabilizing monolayer which is resistant to surface protein deactivation.

The efficacy of the polymer formulation is linked to the characteristics of the self-assembled micelle structure in aqueous conditions. The self-assembly properties of amphiphilic block copolymers in aqueous conditions have been extensively studied over the past several decades. The self-assembly characteristics of a block copolymer (BCP) depends on a variety of factors. BCPs with not too strongly hydrophobic blocks (e.g., Pluronic surfactants from BASF) can be directly dissolved in aqueous conditions. Self-assembly will then occur once a sufficiently high concentration, known as the critical micelle concentration (CMC), is reached. However, for BCPs with strongly hydrophobic blocks which are highly incompatible with water (i.e., those with water-polymer interfacial tensions greater than about 15 mN/m at room temperature such as poly(styrene) (PS)), direct molecular dissolution of the polymer is not possible. Thus, several methods have previously been developed in order to study the self-assembly properties of these systems. As illustrated in FIG. 1A, these methods involve first dissolving the polymer in a non-aqueous common solvent (“co-solvent”) which is compatible with both blocks. Then the solvent conditions are switched from the common solvent to aqueous by either slowly adding water while mixing and then dialyzing to remove the common solvent or by directly dialyzing against water to remove the common solvent. Alternatively, the more volatile co-solvent can be removed using rotary evaporator technique. Both methods provide opportunities for improvement when seeking to scale up production of monodisperse micelles of a BCP system with a strongly hydrophobic block. When using drop-wise addition of water at relatively high concentrations of polymer, the high local concentration of water around the droplet when it contacts the common solvent may cause large aggregates to form due to the incompatibility of the hydrophobic block with water. These large aggregates may remain which may cause the solution to become turbid and may cause size dispersity in the final product.

Additionally, as the solvent composition is changed either from dialysis or water addition the preferred aggregation number of the micelle changes due to the changing interfacial tension between the core and bulk phases. Rearrangement of micelles can occur until a critical water concentration (CWC) is reached. At this composition the exchange of chains from micelles is energetically unfavorable and the micelles become individually isolated (“kinetically frozen”). Both the water addition and direct dialysis method cause micelles to be formed in constantly varying solvent conditions which may leave room for improvement to achieve monodisperse and reproducible micelle systems.

Previous studies by Munk et al. (Makromol. Chem. Macromol. Symp. 58, 195-199, 1992) have shown the approach of mixed solvent micelle formation followed by co-solvent removal via a stepwise dialysis procedure is capable of forming kinetically frozen micelles

### SUMMARY

Hence, this disclosure is proposing a new micelle formulation method (“Equilibration-Nanoprecipitation” or “ENP”) which comprises two distinct steps: (1) forming and equilibrating BCP micelles in a solvent mixture including non-aqueous solvent compositions between about 10 and 90% w/w, and (2) then subsequent dialysis against an aqueous medium to freeze the monodisperse micelle structure and remove or lower the non-aqueous solvent content. Once again, the co-solvent could also be removed via the rotary evaporator technique instead of dialysis. By forming micelles in a uniform solvent composition and allowing time for approximate equilibration and then quickly removing the common solvent past the CWC, a monodisperse kinetically frozen micelle system can be formed.

This disclosure shows that a single-step dialysis approach is able to produce monodisperse micelle systems with greater success than other micelle formation techniques. A stepwise dialysis procedure uses a water/cosolvent mixture bulk reservoir of increasingly higher water contents over time, while this disclosure uses only water as the bulk reservoir. Using a single-step dialysis of a water only reservoir creates a larger composition gradient and increases the rate at which the co-solvent (e.g., acetone) is removed. This may quickly bring the mixture past the CWC and kinetically freeze the micelles in their original equilibrated formation state. Since the micelle size characteristics are relevant for performance properties, control over the dispersity of a given micelle system is a consideration. Thus, the Equilibration-Nanoprecipitation procedure solves the problem of producing monodisperse kinetically frozen micelles from highly hydrophobic amphiphilic BCPs which has not previously been demonstrated. An overview schematic of the procedure is shown in FIG. 1B. The present disclosure is not limited to the specific BCP material (poly(styrene)-b-poly(ethylene glycol) (PS-PEG)) exemplified in this manuscript, but it is broadly applicable to any amphiphilic block copolymers containing strongly hydrophobic blocks.

In one aspect of the invention, a treatment which uses a BCP formulation of PS-PEG for ARDS.

In a further aspect of the invention, it is beneficial to form monodisperse micelles by forming micelles under the same solvent conditions, i.e., water and co-solvent mixtures.

A micelle formulation made by the steps of dissolving amphiphilic block copolymers in a mixed solvent comprising water and a non-aqueous co-solvent, and conducting a single-step dialysis against water or saline or an evaporation process for removal of non-aqueous solvent content in order to produce monodisperse kinetically frozen polymer micelles in aqueous conditions.

A method of forming monodisperse kinetically frozen polymer micelles in aqueous conditions, the method comprising the steps of dissolving amphiphilic block copolymers in a mixed solvent comprising water and a non-aqueous co-solvent to create a micelle solution, and conducting a single-step dialysis against water or saline or an evaporation process to remove the non-aqueous solvent content.

### BRIEF DESCRIPTION OF THE DRAWINGS

The above-mentioned and other features of this disclosure, and the manner of attaining them, will become more apparent and the disclosure itself will be better understood by reference to the following description of embodiments of the disclosure taken in conjunction with the accompanying drawings, wherein:

Unless otherwise stated, a reference to a compound or component includes the compound or component by itself, as well as in combination with other compounds or components, such as mixtures of compounds.

As used herein, the singular forms “a”, “an” and “the” include the plural reference unless the context clearly dictates otherwise.

All publications, patents and patent applications cited herein, whether supra or infra, are hereby incorporated by reference in their entirety to the same extent as if each individual publication, patent or patent application was specifically and individually indicated to be incorporated by reference.

FIG. 1A: Schematic of conventional formulation methods to forming micelles in aqueous environment of amphiphilic BCP with strongly hydrophobic block.

FIG. 1B: Schematic of proposed mixed solvent method to forming micelles in aqueous environment of amphiphilic BCP with strongly hydrophobic block.

FIG. 2A: DLS hydrodynamic diameter size distributions for 100% acetone composition post dialysis.

FIG. 2B: DLS hydrodynamic diameter size distributions for 80% acetone and 20% water mixture composition post dialysis.

FIG. 2C: DLS hydrodynamic diameter size distributions for 70% acetone and 30% water mixture composition post dialysis.

FIG. 2D: DLS hydrodynamic diameter size distributions for 60% acetone and 40% water mixture composition post dialysis.

FIG. 2E: DLS hydrodynamic diameter size distributions for 50% acetone and 50% water mixture composition post dialysis.

FIG. 2F: DLS hydrodynamic diameter size distributions for 40% acetone and 60% water mixture composition post dialysis.

FIG. 3: Surface pressure-area isotherm for micelle systems post dialysis formed at different initial solvent conditions.

FIG. 4A: DLS hydrodynamic diameter size distributions for batch 1 using direct dialysis formulation method.

FIG. 4B: DLS hydrodynamic diameter size distributions for batch 2 using direct dialysis formulation method.

FIG. 4C: DLS hydrodynamic diameter size distributions for batch 3 using direct dialysis formulation method.

FIG. 5: Surface pressure-area isotherms for three different batches using direct dialysis method.

FIG. 6A: DLS hydrodynamic diameter size distributions for batch 1 using the mixed solvent formulation method.

FIG. 6B: DLS hydrodynamic diameter size distributions for batch 2 using the mixed solvent formulation method.

FIG. 6C: DLS hydrodynamic diameter size distributions for batch 3 using the mixed solvent formulation method.

FIG. 7: Surface pressure-area isotherms for three different batches using the mixed solvent formulation method.

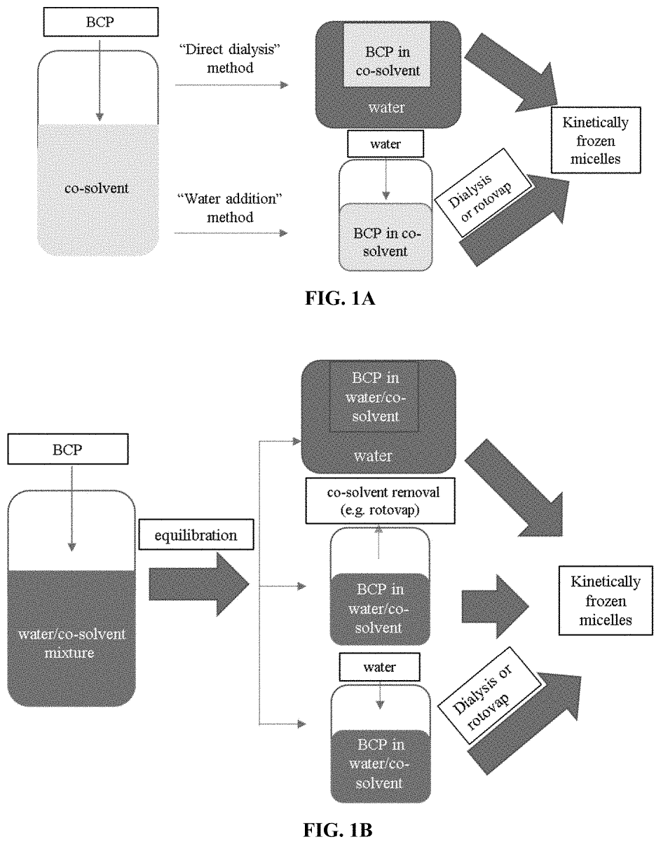

Corresponding reference characters indicate corresponding parts throughout the several views. Although the drawings represent embodiments of the present disclosure, the drawings are not necessarily to scale and certain features may be exaggerated in order to better illustrate and explain the present disclosure.

### DETAILED DESCRIPTION OF THE EXEMPLARY EMBODIMENTS

The embodiments disclosed below are not intended to be exhaustive or limit the disclosure to the precise forms disclosed in the following detailed description. Rather, the embodiments are chosen and described so that others skilled in the art may utilize their teachings.

Experimental Procedures and Materials

Equilibration-Nanoprecipitation (ENP) Micelle Formulation Method. PS-PEG (10 mg) is dissolved in 2 mL mixture of acetone (Sigma-Aldrich) and Milli-Q purified water (18 MΩ·cm resistivity) under sonication. The solution is then repeatedly vortexed and sonicated until the solution appears transparent. The solution is then stored under gentle rocking at room temperature for 24 h to allow for equilibration. Acetone is then removed by dialyzing the 2 mL mixture using Slide-A-Lyzer Mini Dialysis device (20 kDa MWCO) against Milli-Q-purified water for 24 h, replacing the water reservoir at 1, 2, 4 and 6 h time points. The water reservoir is 45 mL.

Direct Dialysis Micelle Formulation Method. The procedure is the same as for Equilibration-Nanoprecipitation procedure except that the polymer is dissolved into acetone only and not an acetone/water mixture.

Polymer Materials. The experiments detailed in this research report are done using PS(5.2 kDa)-PEG(5.5 kDa) purchased from Polymer Source, Inc.

Surface Pressure-Area (SP-A) Isotherms. The surface tension-area isotherms are measured using a KSV Nima Langmuir trough (51 cm×14.5 cm) with double symmetric barriers. The total surface area of the trough is 780 cm², and the subphase volume is 750 mL. A filter paper or platinum Wilhelmy probe is used for surface tension measurements. Micelle samples are spread onto water using a Hamilton micro syringe. The compressions are done at a rate of 3 mm/minute. The temperature of the subphase is held constant at 25° C. using a circulating water bath.

Polymer Micelle Characterizations. The hydrodynamic diameters of the block copolymer micelles are measured at 25° C. by dynamic light scattering (DLS) using a Brookhaven ZetaPALS instrument. The scattering intensities are measured using a 659 nm laser at a scattering angle of 90°. The hydrodynamic diameters were calculated from the measured diffusion coefficients using the Stokes-Einstein equation. The results were averaged over 5 runs.

Results/Discussion

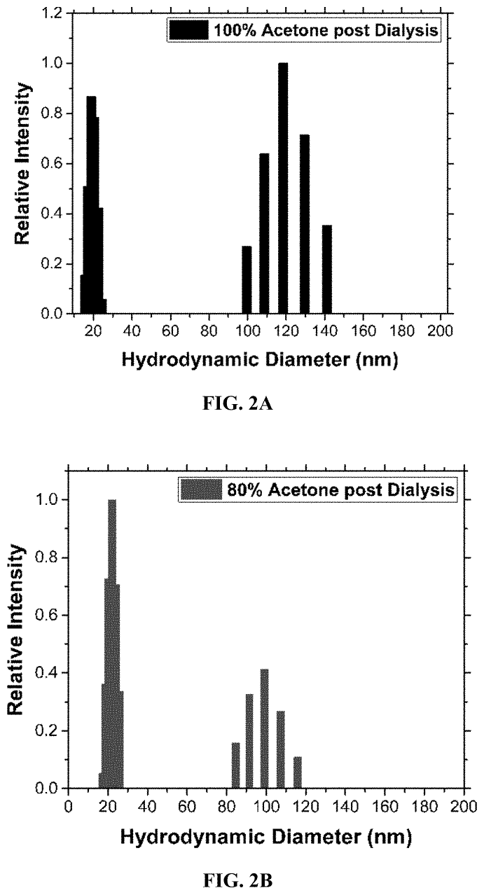

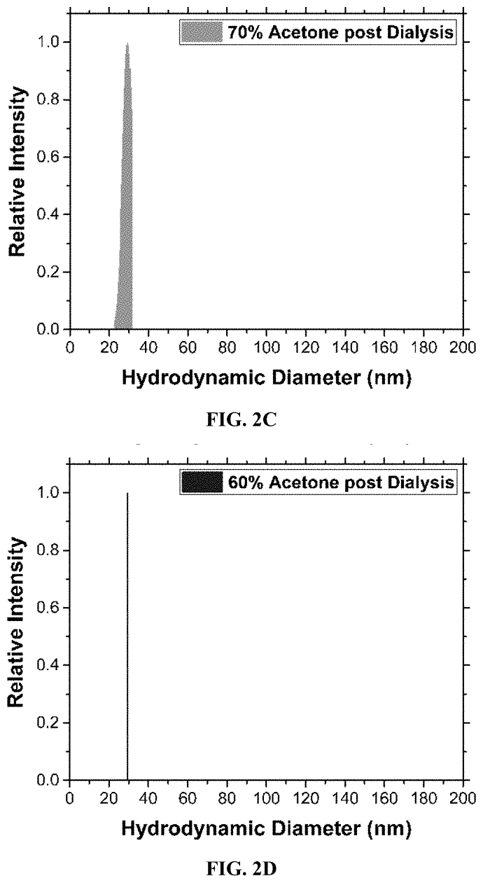

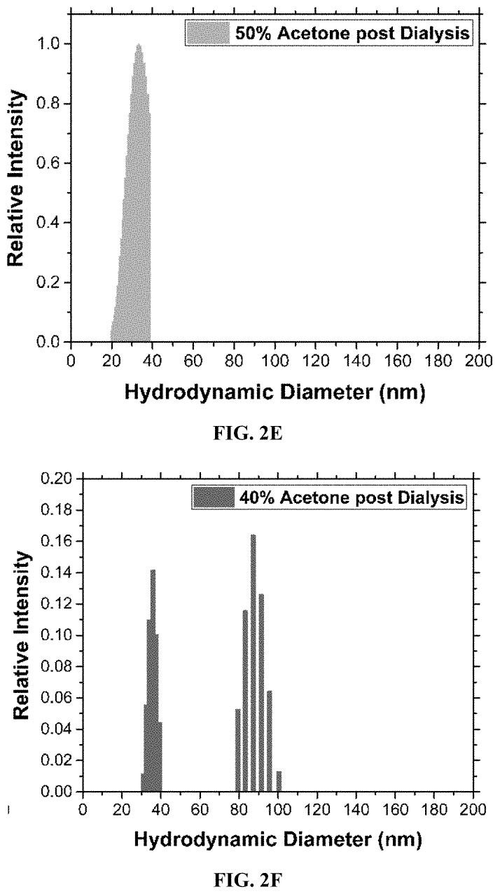

The difference between the directly dialyzing PS(5.2k)-PEG(5.5k) dissolved in acetone (10 mg/mL) and dialyzing micelle systems formed in acetone/water mixtures (10 mg/mL) was demonstrated using DLS and SP-A isotherms. The DLS data in FIG. 2A-FIG. 2F show that for micelle systems formulated from dialyzing dissolved polymer in acetone (“100% Acetone”) two size populations are formed which is also reflected in the high DLS polydispersity (PD) values listed in Table 1. The maximum intensities of the two populations occur at 18.8 nm and 118.0 nm respectively. A similar result is obtained for 20% Acetone system except that the smaller population shows a greater contribution to the size distribution and is centered around a slightly larger value of 22.4 nm. The 70% Acetone, 60% Acetone, and 50% Acetone systems (FIG. 2C, FIG. 2D, and FIG. 2E, respectively) show a narrower distribution with only one size population and smaller PD values. The average hydrodynamic diameter increases with decreasing acetone content which is expected due to the increase in interfacial tension between the core and solvent mixture at higher water contents.

TABLE 1
DLS effective diameter and PD for micelle systems post dialysis formed at various solvent conditions

| | Initial Acetone Composition | | | | | |
|---|---|---|---|---|---|---|
| | 100% | 80% | 70% | 60% | 50% | 40% |
| Effective | 40.1 ± 0.7 | 28.1 ± 0.5 | 28.8 ± 0.2 | 29.6 ± 0.2 | 32.8 ± 0.1 | 52.7 ± 0.2 |
| Diameter | | | | | | |
| (nm) | | | | | | |
| PD | 0.327 ± 0.010 | 0.245 ± 0.003 | 0.068 ± 0.018 | 0.052 ± 0.015 | 0.041 ± 0.005 | 0.163 ± 0.003 |

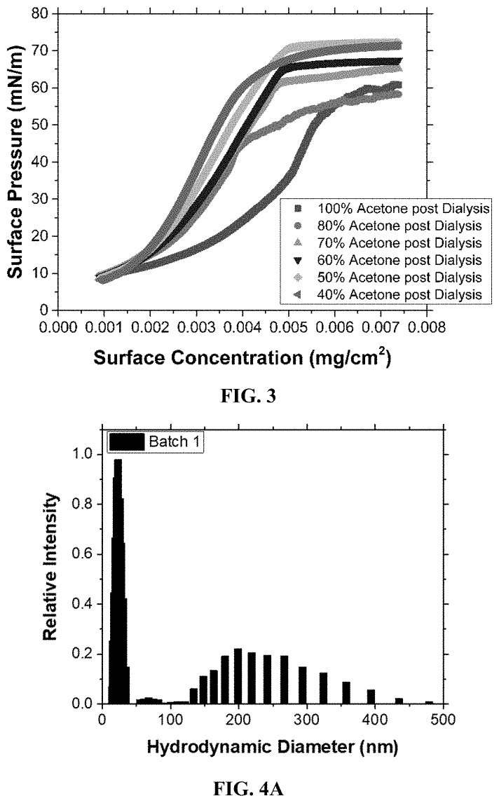

FIG. 3 shows the surface pressure-area (SP-A) isotherms for the various micelle systems post dialysis. The 100% Acetone system produces an isotherm curve which falls much below the other initial solvent compositions until it reaches a similar maximum surface pressure as the 80% Acetone case of around 60 mN/m. The 40% and 50% Acetone cases can achieve nearly complete lowering of the surface tension at the air-water interface as the surface pressure approaches 72 mN/m at high surface concentrations. The demands of the polymer lung surfactant application are such that being able to achieve a surface pressure of greater than about 60 mN/m under high compression is required for proper functioning of the lungs. Thus, the importance of controlling the formulation size characteristics is relevant, and the direct dialysis method leaves room for improvement for this application.

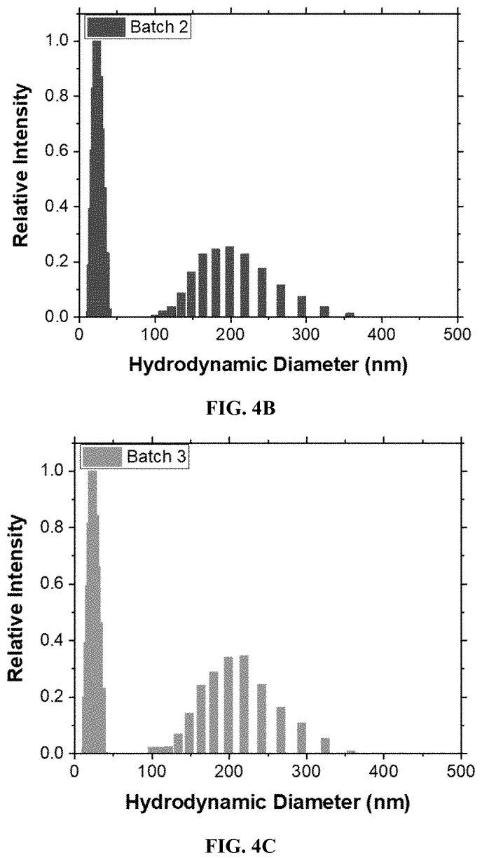

The reproducibility of the direct dialysis method was tested by forming three batches using the same polymer (PS(5.2k)-PEG(5.5k)) and formation conditions (10 mg/mL polymer concentration). The direct dialysis method implies that the polymer is initially dissolved in 100% Acetone then is directly dialyzed. Table 2 and FIG. 4A-FIG. 4C show that there are differences among each batch in the effective diameter, PD, location of the two size populations, and the relative intensities of the smaller and larger populations.

TABLE 2
DLS effective diameter and PD for three different
batches formed using the direct dialysis method.

| Sample | Batch 1 | Batch 2 | Batch 3 |
|---|---|---|---|
| Effective Diameter | 32.7 ± 1.2 | 31.6 ± 1.8 | 35.5 ± 1.5 |
| (nm) | | | |
| PD | 0.208 ± 0.020 | 0.213 ± 0.015 | 0.222 ± 0.015 |

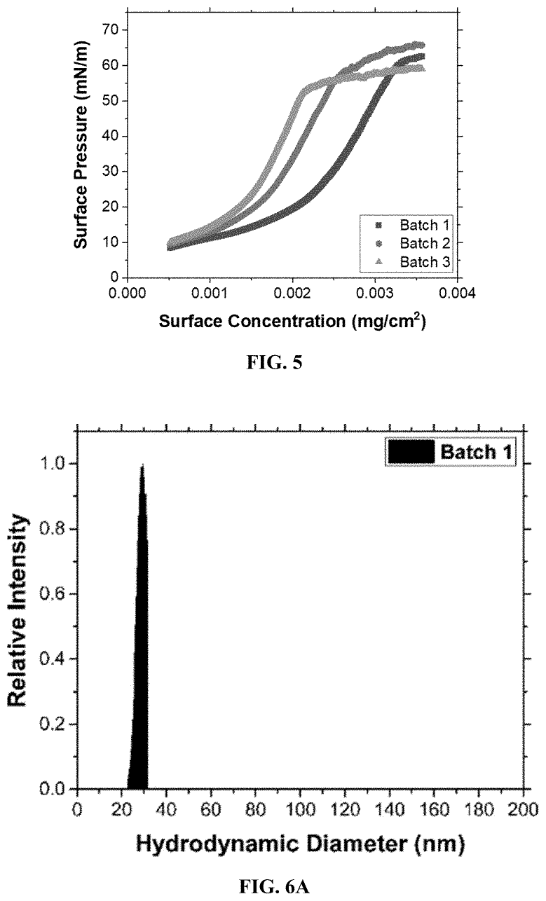

As illustrated in FIG. 5, SP-A isotherm data were collected for each of the three batches, shown in FIG. 4A-FIG. 4C. The differences in DLS data are reflected in the differences in the SP-A isotherm behavior which shows the importance of controlling size characteristics via the formulation procedure. Since the SP-A behavior is directly linked to efficacy, it is relevant that the isotherm behavior is reproducible for different batches.

TABLE 3
DLS effective diameter and PD for three batches
formed using mixed solvent formulation method.

| Sample | Batch 1 | Batch 2 | Batch 3 |
|---|---|---|---|
| Effective Diameter | 28.0 ± 0.1 | 29.4 ± 0.2 | 28.8 ± 0.2 |
| (nm) | | | |
| PD | 0.098 ± 0.015 | 0.121 ± 0.013 | 0.068 ± 0.018 |

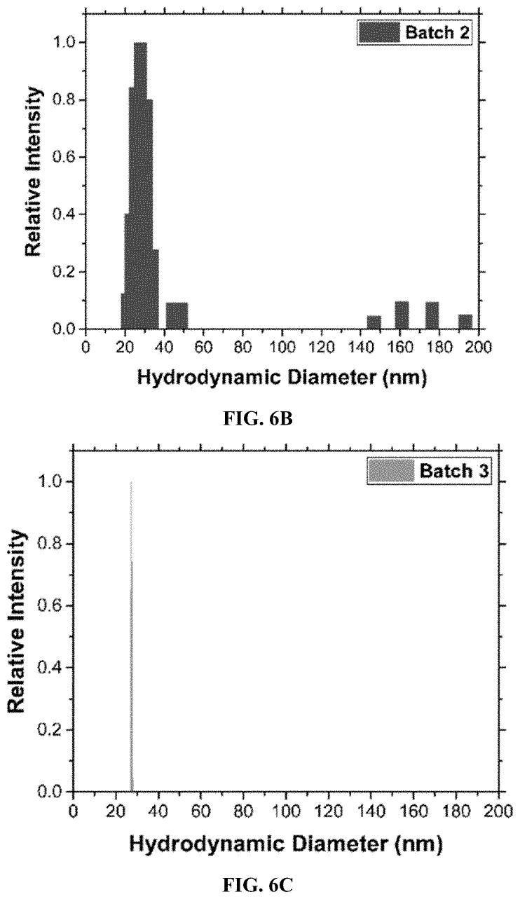

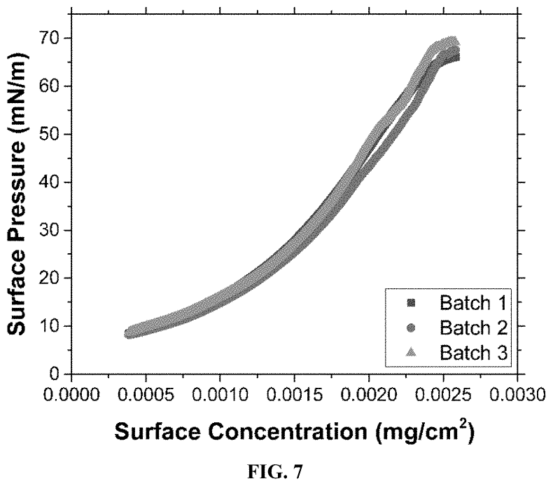

The reproducibility of the mixed solvent method was tested by forming three batches at the 30% acetone solvent mixture condition. The DLS size data post dialysis are shown in Table 3 and FIG. 6A-6C. All three batches show a similar effective diameter and low PD. Batch 2 does show a small contribution of larger sized micelles but the maximum intensities for all in the range of 27-29 nm. The SP-A isotherm data in FIG. 7 reflects the similarity in size distributions as all three isotherms give a very similar shape.

This disclosure is proposing a new micelle formulation method using a mixed solvent approach with a single-step dialysis against water in order to produce monodisperse kinetically frozen polymer micelles in aqueous conditions. This method is an alternative to previous methods involving initial dissolution of BCPs in a non-aqueous co-solvent followed by either direct dialysis or slow addition of water as it initially forms equilibrium micelles in a mixed solvent environment as opposed to an environment containing solvent concentration gradients.

While this disclosure has been described as having an exemplary design, the present disclosure may be further modified within the spirit and scope of this disclosure. This application is therefore intended to cover any variations, uses, or adaptations of the disclosure using its general principles. Further, this application is intended to cover such departures from the present disclosure as come within known or customary practice in the art to which this disclosure pertains.

What is claimed is:

1. A micelle formulation made by a method comprising the steps of:
dissolving amphiphilic block copolymers in a mixed solvent comprising water and a non-aqueous co-solvent, and
conducting a single-step dialysis against water or saline in order to produce monodisperse kinetically frozen polymer micelles with DLS size polydispersities less than about 0.2 in aqueous conditions or
conducting an evaporation process for removal of non-aqueous solvent content in order to produce monodisperse kinetically frozen polymer micelles with DLS size polydispersities less than about 0.2 in aqueous conditions.

2. The micelle formulation of claim 1 wherein the amphiphilic block copolymers include strongly hydrophobic blocks having water-polymer interfacial tensions greater than about 15 mN/m at room temperature.

3. The micelle formulation of claim 1 wherein the amphiphilic block copolymers comprise styrene monomer units.

4. The micelle formulation of claim 1 wherein the mixed solvent includes an overall non-aqueous solvent composition between about 10% and about 90% w/w.

5. The micelle formulation of claim 4 wherein the non-aqueous solvent content is between about 10% and about 90% w/w of the mixed solvent.

6. The micelle formulation of claim 1 wherein the step of dissolving includes the step of forming micelles in a molecularly uniform solvent composition and allowing time for approximate equilibration.

7. A method of forming monodisperse kinetically frozen polymer micelles in aqueous conditions, the method comprising the steps of:
dissolving amphiphilic block copolymers in a mixed solvent comprising water and a non-aqueous co-solvent to create a micelle solution, and
conducting a single-step dialysis against water or saline or
conducting an evaporation process for removal of non-aqueous solvent content.

8. The method of claim 7, wherein the amphiphilic block copolymers comprise styrene monomer units.

9. The method of claim 7, wherein the amphiphilic block copolymers comprise a poly(styrene) block and a poly(ethylene glycol) block.

10. The method of claim 7, wherein the step of dissolving includes the step of:
dissolving PS-PEG block copolymers in a mixture of acetone and water.

11. The method of claim 7, wherein the step of dissolving includes the step of:
sonicating the solution at a certain point during the equilibration process.

12. The method of claim 7, wherein the step of dissolving includes the step of:
mechanically agitating the solution for at least two minutes during the equilibration process.

13. The method of claim 7, wherein the step of conducting a single-step dialysis against water or saline includes the step of:
dialyzing the solution using a dialysis device against a water or saline reservoir for at least 10 minutes.

14. The method of claim 7, wherein the step of conducting a single-step dialysis against water or saline includes the step of:
replacing the aqueous reservoir with fresh water or saline at least once during the process.

15. The method of claim 7, wherein the step of conducting a single-step dialysis against water or saline, wherein the aqueous reservoir is at least larger in volume than the initial micelle solution being dialyzed.
